(19) United States
(12) Patent Application Publication
(10) Pub. No.: US 2025/0278837 A1
(43) Pub. Date: Sep. 4, 2025

(54) MEDICAL IMAGE RECOGNITION APPARATUS, ENDOSCOPE SYSTEM, AND METHOD FOR OPERATING MEDICAL IMAGE RECOGNITION APPARATUS

(71) Applicant: FUJIFILM Corporation, Tokyo (JP)

(72) Inventor: Masahiro KITAHARA, Kanagawa (JP)

(73) Assignee: FUJIFILM Corporation, Tokyo (JP)

(21) Appl. No.: 19/211,266

(22) Filed: May 18, 2025

Related U.S. Application Data
(63) Continuation of application No. PCT/JP2023/038685, filed on Oct. 26, 2023.

(30) Foreign Application Priority Data
Nov. 22, 2022 (JP) 2022-186791

(51) Int. Cl. G06T 7/00; A61B 1/00; A61B 1/04; G06T 7/11

(52) U.S. Cl. CPC G06T 7/0014; A61B 1/000096; A61B 1/0004; A61B 1/00043; A61B 1/04; G06T 7/11; G06T 2207/10068; G06T 2207/20081; G06T 2207/20084; G06T 2207/30104

(57) ABSTRACT

A processor configured to acquire a medical image; set a reference region and an evaluation region different from the reference region in an organ portion of the medical image; input color information of the medical image of the reference region and the medical image of the evaluation region to a trained classifier; output an evaluation result of a blood flow attribute of the evaluation region with respect to a blood flow attribute of the reference region; and perform control to display an evaluation result of the blood flow attribute of the evaluation region.

### CROSS-REFERENCE TO RELATED APPLICATIONS

This application is a Continuation of PCT International Application No. PCT/JP2023/038685 filed on 26 Oct. 2023, which claims priority under 35 U.S.C § 119 (a) to Japanese Patent Application No. 2022-186791 filed on 22 Nov. 2022. The above application is hereby expressly incorporated by reference, in its entirety, into the present application.

### BACKGROUND OF THE INVENTION

### 1. Field of the Invention

The present disclosure relates to a medical image recognition apparatus, an endoscope system, and a method for operating the medical image recognition apparatus.

### 2. Description of the Related Art

In the medical field, a main complication occurring in an intestinal resection technique is suture failure. When stool leaking from seams results in peritonitis, re-surgery is necessary. The frequency of occurrence of suture failure is also high at approximately 1.5% in colon cancer surgery and at approximately 10% in rectal cancer surgery. One of the causes of suture failure is that blood does not flow through a sutured region. For this reason, by checking the state of blood flow in tissue before suturing, it is expected to reduce the risk of post-operative complications.

Examples of the method of blood flow confirmation include an indocyanine green (ICG) fluorescence method in which a fluorescent dye is intravenously injected to infiltrate tissues or capillaries and then infrared light observation is performed. It is also known that since the blood flow state and the tissue oxygen saturation are not uncorrelated, there are cases in which the presence or absence of blood flow can be visualized by using an oxygen saturation imaging technique for quantifying the oxygen saturation of tissue (JP2018-027401A).

In JP2018-027401A, specifically, an observation target is classified by the amounts of blood, the levels of oxygen saturation, and a threshold value on the basis of spectral images, and a diagnostic support image is generated in which a high-blood-amount and low-oxygen region and a low-blood-amount and low-oxygen region are represented by density or by color in accordance with the oxygen saturation. In JP2005-192880A, clustering is performed using the K-means method on the basis of the distances between the average values of a cluster indicating normal mucosa and a cluster indicating abnormal mucosa (redness, bleeding, or discoloration) in a feature space defined by chromaticities.

### SUMMARY OF THE INVENTION

However, blood flow confirmation using the ICG fluorescence method has problems in that it is necessary to wait for infiltration of a drug and that it is invasive due to intravenous injection of the drug, and the output of oxygen saturation imaging described in JP2018-027401A, in which the oxygen saturation is calculated to determine the presence or absence of blood flow and a threshold value is used to determine a normal region and an ischemic region, lacks robustness against external factors such as inter-patient differences and light sources. In JP2005-192880A, an abnormal finding such as mucosal bleeding is detected by using chromaticities, but a normal region and an ischemic region are not classified.

Accordingly, it is desired to implement a low-invasive and ready-to-use image processing technique that operates robustly against external factors to distinguish between a normal blood flow region and an ischemic region appearing in an intraoperative image.

An object of the present disclosure is to provide a medical image recognition apparatus, an endoscope system, and a method for operating the medical image recognition apparatus that operate robustly against external factors and are capable of distinguishing a medical image having a normal blood flow region and an ischemic region in a low-invasive and ready-to-use manner.

A medical image recognition apparatus according to an examplary embodiment of the invention includes a processor configured to acquire a medical image, set a reference region and an evaluation region different from the reference region in an organ portion of the medical image, input color information of the reference region and color information of the evaluation region to a trained classifier, output an evaluation result of a blood flow attribute of the evaluation region with respect to a blood flow attribute of the reference region, and perform control to display an evaluation result of the blood flow attribute of the evaluation region.

It is preferable to set the reference region by using at least one of manual setting by a user or automatic recognition.

It is preferable to recognize that in the medical image, a region for which a difference from the color information of the reference region is within a certain range has a blood flow attribute equal to the blood flow attribute of the reference region, and recognize that in the medical image, a region for which a difference from the color information of the evaluation region is within a certain range has a blood flow attribute equal to the blood flow attribute of the evaluation region.

It is preferable to calculate, for the medical image input to the trained classifier, a probability that the blood flow attribute of the reference region and the blood flow attribute of the evaluation region are equal, and output that the blood flow attribute of the reference region and the blood flow attribute of the evaluation region are equal in a case where the probability is greater than or equal to a preset threshold value.

It is preferable to extract pixel values of the reference region and the evaluation region as the color information, and output an evaluation result acquired by converting the pixel values into feature values.

It is preferable to apply a grid to the medical image and set the evaluation region from regions segmented by the grid.

It is preferable to visualize and display the blood flow attribute of the reference region together with the evaluation result of the blood flow attribute of the evaluation region.

It is preferable to set the reference region and the evaluation region in one medical image.

It is preferable to set a plurality of the evaluation regions from one medical image, and display evaluation results of blood flow attributes of the plurality of the evaluation regions.

It is preferable to determine whether the evaluation region is a normal region in which blood flow is normal or an ischemic region in which blood flow is ischemic, by using the blood flow attribute of the reference region as a reference.

It is preferable to use the trained classifier trained using a learning image associated with an image region for each blood flow attribute.

It is preferable to use the trained classifier trained using average pixel values of small regions randomly extracted from the normal region and the ischemic region in the learning image.

It is preferable to display the medical image having the normal region and the ischemic region in preference to the medical image having either the normal region or the ischemic region.

An endoscope system according to an examplary embodiment of the invention includes the medical image recognition apparatus, an endoscope that acquires the medical image, a display that displays the medical image and the evaluation result of the blood flow attribute of the evaluation region, and user input means for accepting manual setting by a user.

A method for operating a medical image recognition apparatus according to an examplary embodiment of the invention includes a step of acquiring a medical image, a step of setting a reference region and an evaluation region different from the reference region in an organ portion of the medical image, a step of inputting color information of the reference region and color information of the evaluation region to a trained classifier, a step of outputting an evaluation result of a blood flow attribute of the evaluation region with respect to a blood flow attribute of the reference region, and a step of performing control to display an evaluation result of the blood flow attribute of the evaluation region.

According to the present disclosure, it is possible to operate robustly against external factors and identify a medical image having a normal blood flow region and an ischemic region in a low-invasive and ready-to-use manner.

### BRIEF DESCRIPTION OF THE DRAWINGS

FIG. 1 is an explanatory diagram illustrating a configuration of a medical image recognition system;

FIG. 2 is a block diagram illustrating functions of a medical image recognition apparatus;

FIG. 3A is an explanatory diagram of an imaging site of a photographic subject, and FIG. 3B is an explanatory diagram of a medical image having an organ portion, which is acquired from the photographic subject;

FIG. 4 is an explanatory diagram of setting of a reference region and a search region by using region setting;

FIG. 5A is an explanatory diagram of a medical image in which the reference region is a normal region and the search region is an ischemic region, and FIG. 5B is an explanatory diagram of conversion of pixel values of the normal region and the ischemic region into values in a color space;

FIG. 6A is an explanatory diagram of a medical image that is affected by a disturbance and in which the reference region is a normal region and the search region is an ischemic region, and FIG. 6B is an explanatory diagram of the color space in which the pixel values of the normal region and the ischemic region are shifted by the disturbance;

FIG. 7 is an explanatory diagram of a relationship between blood flow attributes of the reference region and the search region, which is inferred by inputting medical images to a classifier;

FIG. 8 is an explanatory diagram of calculation of the probability that the blood flow attributes are equal by using pixel values extracted from a medical image input to the classifier;

FIG. 9 is an explanatory diagram of recognition of a region having a blood flow attribute equal to that of the reference region and a region having a blood flow attribute equal to that of the search region when the reference region and the search region are classified as having different blood flow attributes;

FIG. 10 is an explanatory diagram of recognition of a region having a blood flow attribute equal to those of the reference region and the search region when the reference region and the search region are classified as having the same blood flow attribute;

FIG. 11 is an explanatory diagram of a screen display of a medical image having an organ portion in which the normal region and the ischemic region are recognized;

FIG. 12 is an explanatory diagram of a screen display of a medical image in which the reference region and the search region have the same blood flow attribute;

FIG. 13 is an explanatory diagram of training of a CNN using input image sets each being a set of three images;

FIGS. 14A, 14B, and 14C are explanatory diagrams of patterns in which a negative region in a negative image is an ischemic region, has two normal regions, and has two ischemic regions, respectively;

FIGS. 15A, 15B, and 15C are explanatory diagrams of extraction of pixel value information to be input to the CNN, from an anchor image, a positive image, and a negative image of the input image sets, respectively;

FIG. 16 is an explanatory diagram of training of the CNN by inputting pixel values extracted from the input image sets;

FIGS. 17A, 17B, and 17C are explanatory diagrams of conversion of pixel values of a positive image, a negative image having a normal region, and a negative image having an ischemic region, respectively, into feature values;

FIG. 18 is an explanatory diagram of four patterns of medical images to be input during inference;

FIG. 19 is an explanatory diagram of feature value conversion for the four patterns of medical images during inference and inference results; and

FIG. 20 is a flowchart illustrating the flow of a series of processes according to the present disclosure.

### DESCRIPTION OF THE PREFERRED EMBODIMENTS

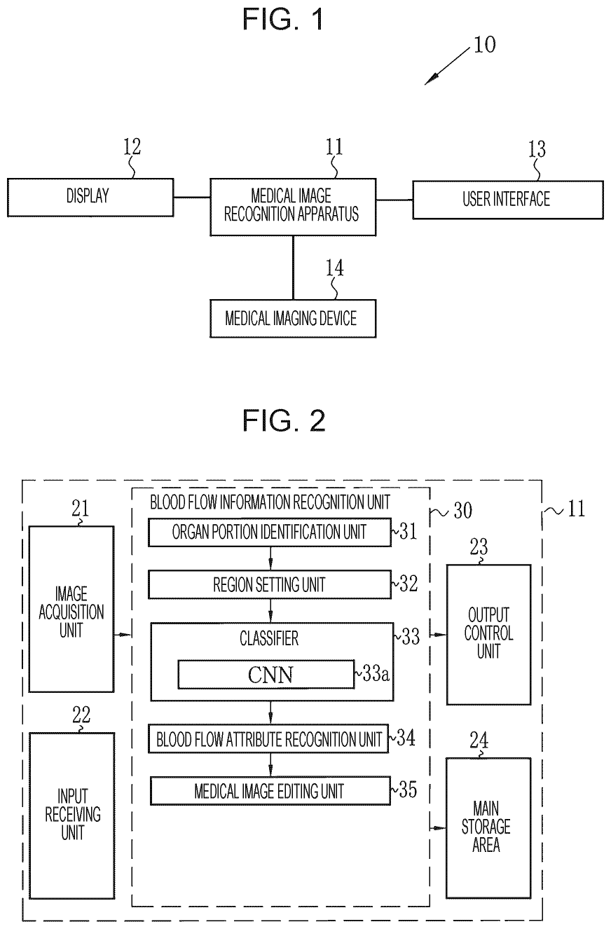

FIG. 1 is a diagram illustrating a configuration of a medical image recognition system 10 having a medical image recognition apparatus 11 according to an embodiment of the present disclosure. The medical image recognition system 10 has the medical image recognition apparatus 11, a display 12, a user interface (UI) 13 and a medical imaging device 14. The medical image recognition apparatus 11 is electrically connected to the display 12, the user interface 13, and the medical imaging device 14. In the medical image recognition apparatus 11, a program related to processing such as image processing is stored in a program memory (not illustrated).

The medical imaging device 14 is a device capable of transmitting and receiving data to and from the medical image recognition apparatus 11 and storing the data, and acquires a medical image obtained by imaging an organ portion. In an intestinal resection technique, an image of an intestinal tract to be operated on or a candidate thereof is captured as an organ portion. The medical imaging device 14 is an imaging device such as a rigid endoscope such as a laparoscope that captures an image of an organ portion, or a surgical camera that captures an image of an organ portion during laparotomy. The medical image recognition apparatus 11 may be electrically connected to a recording medium such as a database that stores acquired medical images, instead of the medical imaging device 14, to indirectly acquire a medical image. The medical images are preferably stored in the database by using an internal solid state drive (SSD). Instead of an SSD, a recording medium such as a Universal Serial Bus (USB) memory or a hard disc drive (HDD) may be used. When an endoscope is used as the medical imaging device 14, the medical image recognition system 10 also functions as an endoscope system.

As illustrated in FIG. 2, in the medical image recognition apparatus 11, a program in the program memory is operated by a central control unit (not illustrated), which is constituted by an image control processor, to implement the functions of an image acquisition unit 21, an input receiving unit 22, an output control unit 23, a main storage area 24, and a blood flow information recognition unit 30. The image acquisition unit 21 receives a medical image captured by the medical imaging device 14 and transmits the medical image to the blood flow information recognition unit 30. The input receiving unit 22 is connected to the user interface 13 and accepts a user input via the user interface 13. The output control unit 23 performs control to display the medical image on the display 12 and control to transmit the medical image to an external device. The main storage area 24 stores a medical image with associated blood flow information. When the functions of the blood flow information recognition unit 30 are implemented, the functions of an organ portion identification unit 31, a region setting unit 32, a classifier 33 including a convolutional neural network (CNN) 33a, a blood flow attribute recognition unit 34, and a medical image editing unit 35 are implemented. The blood flow information is information on a blood flow attribute indicating whether blood flow is normal or ischemic and on an image region for each blood flow attribute.

The organ portion identification unit 31 determines whether an acquired medical image 40 has an organ portion T, and identifies the shape of the organ portion T when the medical image 40 has the organ portion T. The region setting unit 32 selects a reference region in the organ portion T identified in the medical image 40. After setting the reference region, the region setting unit 32 sets a search region different from the reference region from the organ portion T in the medical image 40. The region setting unit 32 may set the reference region and the search region in one medical image 40, or set each of the reference region and the search region in a different medical image 40. The search region is preferably comprehensively extracted from the medical image 40. The CNN 33a included in the classifier 33 is a computer algorithm implemented by a neural network that performs machine learning using input learning data.

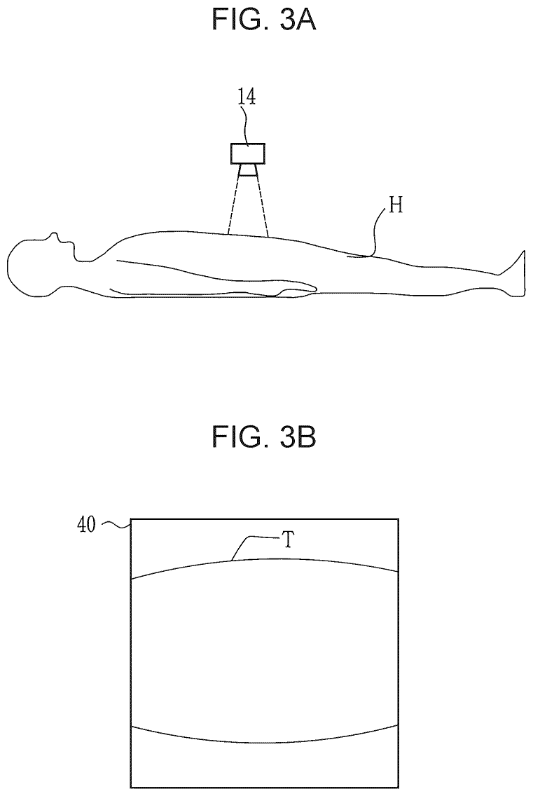

As illustrated in FIGS. 3A and 3B, the medical imaging device 14 captures an image of an abdominal incision site or the like of a photographic subject H (FIG. 3A) and transmits the captured image to the medical image recognition apparatus 11. The medical image recognition apparatus 11 acquires the medical image 40, which is an intraoperative image having the organ portion T, from the image acquisition unit 21 (FIG. 3B). The organ portion T preferably occupies half or more of the area of the medical image 40. The medical image 40 is acquired as a color image. The photographic subject H is a patient or the like scheduled to undergo intestinal resection.

The classifier 33 is a classifier trained on the blood flow information of the organ portion T and performs two-class classification to classify whether the attributes of the blood flow state (blood flow attributes) in the reference region and the search region set by the region setting unit 32 are the same. Specifically, blood flow information of the organ portion T in a learning image is learned in advance, and color information in medical images 40 having the reference region and medical images 40 having the search region is input to the trained classifier 33 to infer the blood flow information of the organ portion T in the medical image 40. Preferably, the CNN 33a learns the relationship between a normal region and an ischemic region in a medical image by using average pixel values of small regions randomly extracted from the normal region and the ischemic region in learning images and performs classification by a method using a triplet loss. The triplet loss will be described below.

The blood flow attribute recognition unit 34 recognizes blood flow attributes of the reference region and the search region in the organ portion T of the medical image 40 on which two-class classification processing has been performed, by determining a normal region in which blood flow is normal or an ischemic region in which blood flow is ischemic. Specifically, the blood flow attribute recognition unit 34 identifies the blood flow attribute of the reference region by using means described below or the like, and determines, based on the blood flow attribute of the reference region, whether the search region is a normal region in which the blood flow in the search region is normal or an ischemic region in which the blood flow in the search region is ischemic. That is, the search region is a region for evaluating the blood flow attribute using the blood flow attribute of the identified reference region as a reference. The blood flow attribute recognition unit 34 further recognizes regions that are the normal region and the ischemic region other than the reference region and the search region in the organ portion T.

The medical image editing unit 35 performs editing to visualize the evaluation result of the blood flow attribute of at least one of the reference region or the search region recognized by the blood flow attribute recognition unit 34 in the organ portion T of the medical image 40 by creating a schematic diagram, displaying the evaluation result in a superimposed manner on the medical image 40, or the like. For example, the normal region is filled with red color, the ischemic region is filled with blue color, and boundaries (demarcation lines) of the regions are displayed. The medical image 40 and the evaluation result thereof are displayed on the display 12 through the output control unit 23. The medical image 40 for which the blood flow information has been edited is stored in the main storage area 24.

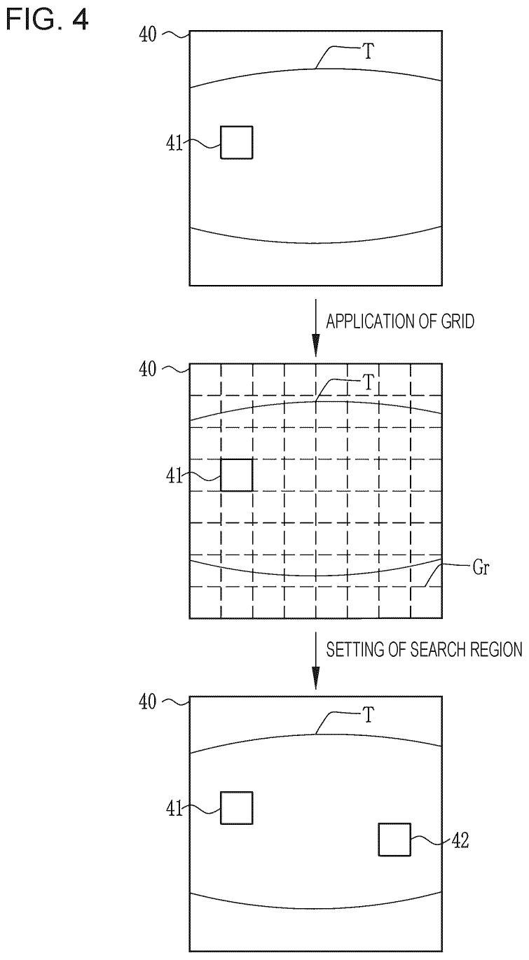

A description will be given of a case where, as illustrated in FIG. 4, the region setting unit 32 sets a reference region 41 and a search region 42 in one single medical image 40. In the medical image 40, the reference region 41 is set from the organ portion T. The reference region 41 may be set as desired by manual selection by the user, namely, selection through the user interface 13, or may be set by automatic recognition. After the reference region 41 is set, a lattice-based grid Gr is applied to the medical image 40 to comprehensively extract the search region 42. The search region 42 is a region that does not include the reference region 41 in the organ portion T among regions into which the medical image 40 is equally divided by the grid Gr. The search region 42 is preferably a region including a pixel value farthest from the reference region 41 in the organ portion T. The medical image 40 is transmitted to the classifier 33 in association with information on the reference region 41 and the search region 42 that have been set. Not one, but multiple search regions 42 may be set in one medical image 40. In this case, the search regions 42 are regions having a threshold value away from the reference region 41 by a certain value or more and not overlapping each other.

Preferably, the lattices of the grid Gr to be applied to the medical image 40 are not always arranged at equal intervals, but are set in accordance with actual dimensions. For example, when the medical imaging device 14 is a zoom scope, the lattices of the grid Gr are set to be coarser, and when the medical imaging device 14 is a rigid endoscope and captures a distant subject, the lattices of the grid Gr are set to be finer. In addition to one search region 42, a second search region (not illustrated) at a position a certain distance away from both the reference region 41 and the search region 42 may be extracted. In this case, classification is performed on the relationship between the blood flow attributes of the pair of the reference region 41 and the search region 42 and the pair of the reference region 41 and the second search region.

When the reference region 41 and the search region 42 are to be set in different medical images 40, the medical image 40 in which the reference region 41 is set is held, and color information such as the pixel values of the reference region 41 is acquired. The acquired color information is provided to the medical image 40 in which the search region 42 is to be set by applying the grid Gr.

In the two-class classification processing, classification is performed, based on the relationship of color information such as pixel values, to determine whether the blood flow attributes in the reference region 41 and the search region 42 are identical. A region with “normal” blood flow attribute is fully supplied with blood, and a region with “ischemic” blood flow attribute is insufficiently supplied with blood. The ischemic region has a lower proportion of hemoglobin, which is a red pigment, than the normal region and can be identified on the basis of a difference in the configuration of color information between the ischemic region and the normal region. The ischemic region has less reddish color and relatively bluish white color compared to the normal region.

When three types of pixel values, namely, an R pixel value, a G pixel value, and a B pixel value, are used as color information, the reference region 41 and the search region 42 can be represented as three-dimensional distributions in a color space whose axes represent the R pixel value, the G pixel value, and the B pixel value.

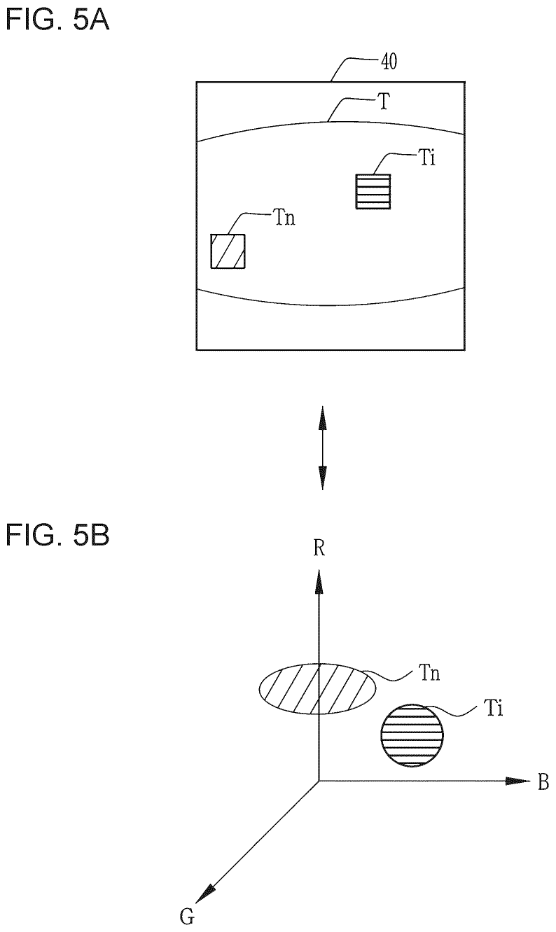

As illustrated in FIGS. 5A and 5B, when one of the reference region 41 and the search region 42 in the organ portion T is a normal region Tn having “normal” blood flow attribute and the other is an ischemic region Ti having “ischemia” blood flow attribute (FIG. 5A), two types of pixel value distributions are present in the color space. Since the normal region Tn contains a larger amount of blood than the ischemic region Ti, the normal region Tn has a distribution with a larger number of R pixels and a smaller number of B pixels (FIG. 5B). The distributions in the color space may be the actual measurement values of the R pixel value, the G pixel value, and the B pixel value in the reference region 41 and the search region 42, or may be the ratios of the respective pixel values.

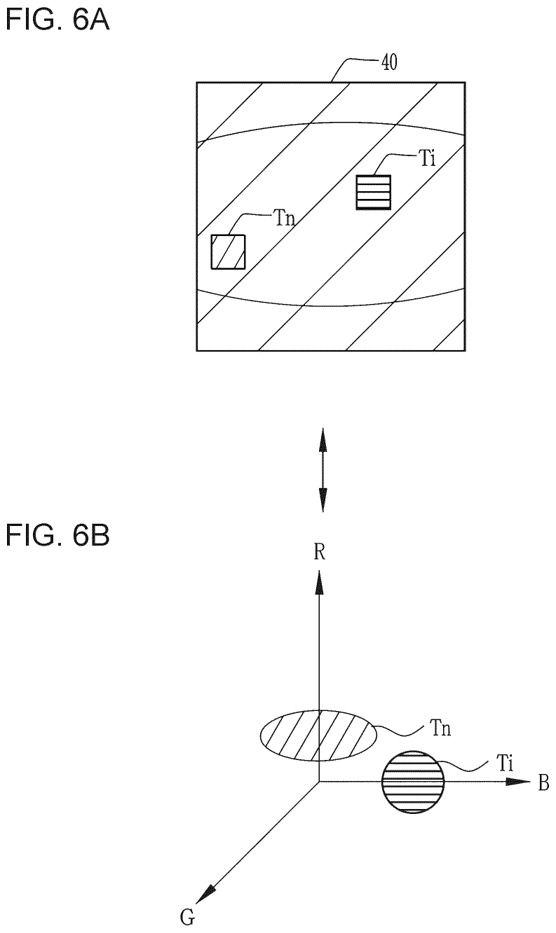

As illustrated in FIGS. 6A and 6B, even medical images 40 obtained by imaging the same organ portion T may have greatly different color information due to the effect of a disturbance such as a light source. The disturbance causes a shift in the pixel value distributions in the color space. For example, the medical image 40 in FIG. 5A described above is captured under white light conditions, and FIG. 6A illustrates a medical image 40 captured under yellow light conditions, for example. In FIGS. 6A and 6B, an image of the same organ portion T as that FIGS. 5A and 5B is captured under the same imaging conditions as those in FIGS. 5A and 5B except for the light source. In this case, as illustrated in FIG. 6B, the pixel values for blue, which is the complementary color of yellow, in the reference region 41 and the search region 42, that is, the B pixel values, are shifted to lower values than those in FIG. 5B throughout the entire image, and the G pixel values, which are the pixel values for green, and the R pixel values, which are the pixel values for red, are shifted to higher values than those in FIG. 5B.

The disturbance affects the entire image, and the pixel values or the pixel ratios of the reference region 41 and the search region 42 shift in a similar manner. However, the relationship remains unchanged. Thus, the two-class classification performed by the classifier 33 to classify the relationship between the blood flow attributes by using the relationship in color information between the two regions is not affected by the disturbance.

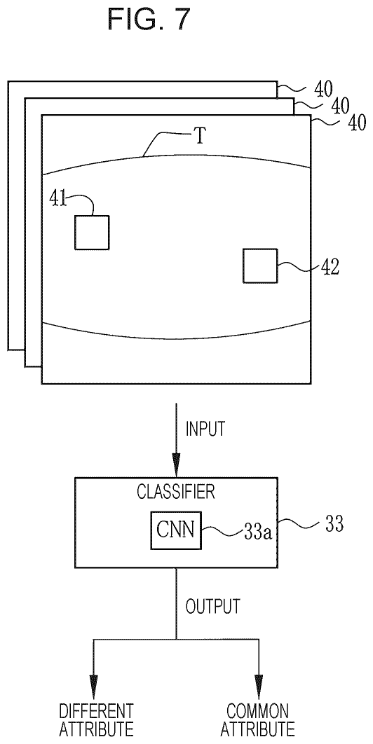

As illustrated in FIG. 7, the classifier 33 performs the two-class classification processing using a preset threshold value for an inference result of the CNN 33a indicating a probability that the reference region 41 and the search region 42 in the medical image 40 have a common attribute to classify whether the reference region 41 and the search region 42 have a common blood flow attribute. The two-class classification processing is executed by inputting the medical image 40, in which the reference region 41 and the search region 42 are set, into the classifier 33 that includes the CNN 33a that has learned the color information of the normal region In and the ischemic region Ti in advance and in which a threshold value is set for the probability output from the CNN 33a. For example, if the probability is greater than or equal to a preset threshold value, the reference region 41 and the search region 42 are classified as having the same blood flow attribute. The classifier 33 extracts the pixel values in the reference region 41 and the pixel values in the search region 42 of the input medical image, and inputs the extracted pixel values to the CNN 33a.

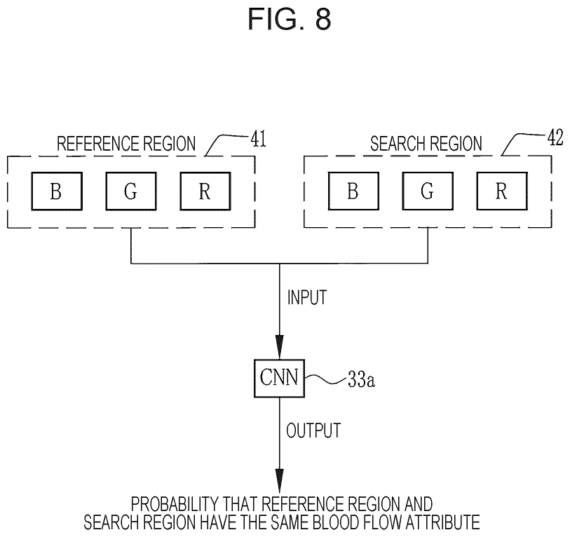

As illustrated in FIG. 8, the CNN 33a accepts input of the B pixel value, the G pixel value, and the R pixel value of the reference region 41 and the B pixel value, the G pixel value, and the R pixel value of the search region 42 in the medical image 40 acquired by the classifier 33, infers a probability that the reference region 41 and the search region 42 have the same blood flow attribute, and outputs the probability. The CNN 33a classifies, based on the probability, whether the two regions have a common attribute or different attributes. That is, the medical image 40 having a pair of pixel information of the reference region 41 and pixel information of the search region 42 is input to the classifier 33, and an output recognition result indicating the relationship between the reference region 41 and the search region 42 is acquired. The acquired recognition result is preferably held in association with the medical image 40. The classifier 33 may output the recognition result including the evaluation value of the blood flow state.

In response to acquiring the result of two-class classification from the classifier 33, the blood flow attribute recognition unit 34 performs attribute determination processing for determining whether the reference region 41 or the search region 42 of the medical image 40 is the normal region In or the ischemic region Ti. The attribute determination processing may be performed by using, for example, a trained classifier different from the classifier 33 or through the user's visual annotation.

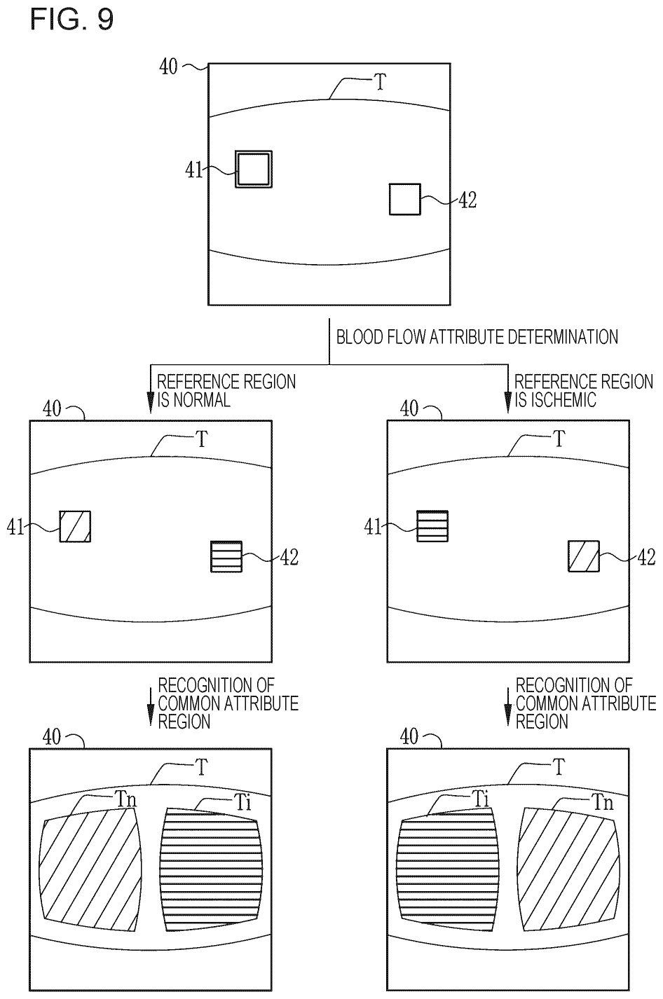

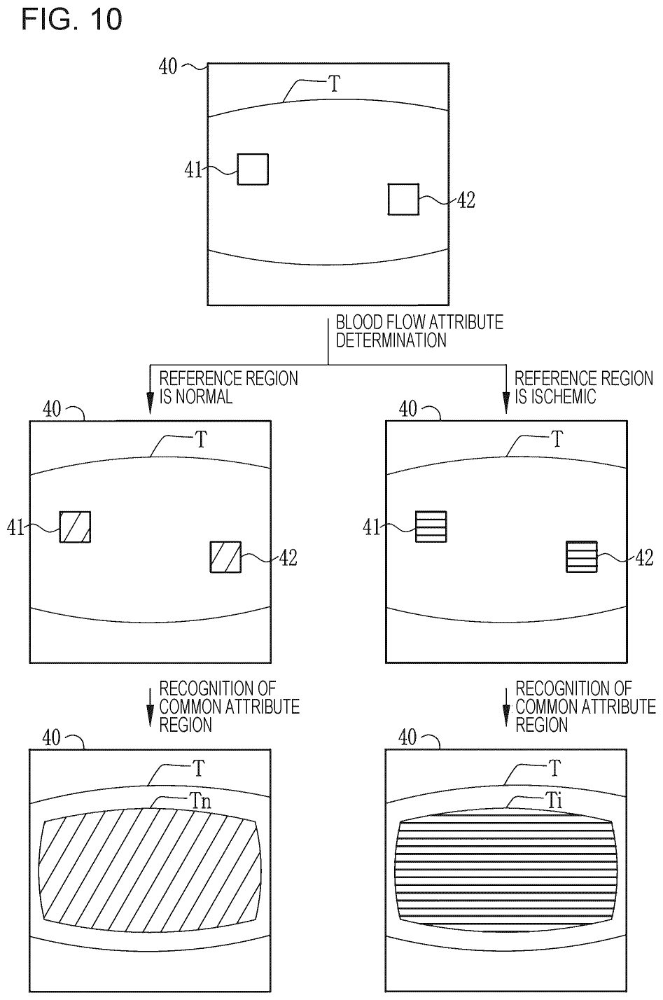

As illustrated in FIGS. 9 and 10, the blood flow attribute recognition unit 34 recognizes blood flow attributes in the reference region 41 and the search region 42, which are classified by the classifier 33 to determine whether the blood flow attributes are common or different. Since the relationship between the blood flow attributes of the reference region 41 and the search region 42 is classified by the classifier 33, one of the blood flow attributes can be identified, thereby enabling the other blood flow attribute to be determined. Further, the blood flow attribute recognition unit 34 also recognizes the blood flow attributes of other regions in the organ portion T on the basis of the pixel value information of the reference region 41 and the search region 42. For example, if a difference in pixel value distribution from the reference region 41 or the search region 42 falls within a certain range, the blood flow attributes are recognized to be identical. The pixel value distributions of the normal region In and the ischemic region Ti are set so as not to overlap.

A description will be given of a case where, as illustrated in FIG. 9, the classifier 33 classifies the reference region 41 and the search region 42 as having different blood flow attributes. When the blood flow attribute of the reference region 41 is recognized as being normal, the blood flow attribute of the search region 42 is determined to be ischemic. Further, a region for which a difference in color information from the reference region 41 and the search region 42 is within a certain range, that is, a common attribute region, is recognized in the organ portion T. That is, the pixel values of the entire organ portion T are acquired, and it is recognized whether each of the pixel values is in the range of the normal region In, the range of the ischemic region Ti, or neither. As a result, the blood flow attributes in the entire organ portion T are identified. When the blood flow attribute of the reference region 41 is recognized as being ischemic, the blood flow attribute of the search region 42 is recognized as being normal, and the blood flow attributes in the entire organ portion T are identified in a similar manner.

A description will be given of a case where, as illustrated in FIG. 10, the classifier 33 classifies the reference region 41 and the search region 42 as having a common blood flow attribute. When the blood flow attribute of the reference region 41 is recognized as being normal, the blood flow attribute of the search region 42 is recognized as also being normal. Further, representative values of the pixel values of the two regions are determined, and a region for which the difference in color information in the organ portion T from the representative values of the color information of the reference region 41 and the search region 42 falls within a certain range is recognized. That is, the pixel values of the entire organ portion T are acquired, and a region for which a difference from the representative values is within a certain range is recognized as the normal region Tn. Also when the blood flow attributes of the reference region 41 and the search region 42 indicate the ischemic region Ti, a region having pixel value information within a certain range is determined in a similar manner.

In the recognition of the common attribute region, a region within a certain range with respect to the color information of the normal region In or the ischemic region Ti can be a region having pixel values within a certain range from the representative value of the pixel value distribution of the normal region Tn or the ischemic region Ti. For example, representative values of the pixel value distributions in the normal region Tn are represented by Rn for the R pixel value, Gn for the G pixel value, and Bn for the B pixel value. In this case, a region having pixel values satisfying Rn×0.9<Rn<Rn×1.1, Gn×0.95<Gn<Gn×1.05, and Bn×0.95<Bn<Bn×1.05 is recognized as having the same blood flow attribute. Since the organ portion T is greatly affected by red color, which is the color of blood, the range of R pixel values is preferably set to be wider than the range of G pixel values and the range of B pixel values. Also when representative values for the ischemic region Ti are represented by Ri, Gi, and Bi, a certain range is set in a manner similar to that for the normal region Tn. Alternatively, a range for the pixel value distributions in the normal region In may be identified, and a region having pixel values that match the pixel value distributions may be recognized as having the same blood flow attribute.

The medical image 40 that holds the identified information on the normal region Tn and the ischemic region Ti is transmitted to the medical image editing unit 35. The medical image editing unit 35 performs editing and the like to visualize the recognized normal region Tn and ischemic region Ti. For example, the medical image editing unit 35 performs editing such that the normal region Tn is in red and the ischemic region Ti is in blue. The blood flow attribute of the search region 42 instead of the reference region 41 may be recognized, and the blood flow attribute of the reference region 41 may be identified in accordance with the recognition result.

When the organ portion T has the normal region Tn and the ischemic region Ti, a demarcation line (not illustrated) may be displayed in a superimposed manner on the medical image 40 to allow the boundary between two regions to be visually recognized. The demarcation line facilitates determination of a site to be excised in an intestinal resection technique.

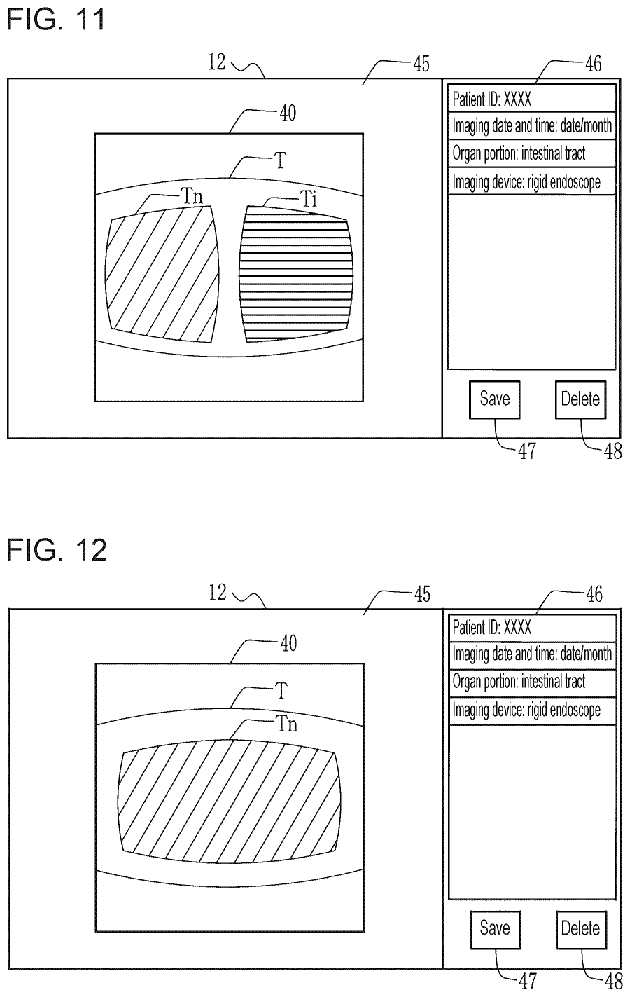

As illustrated in FIG. 11, when the organ portion T in the medical image 40 has the normal region Tn and the ischemic region Ti, the medical image 40 is displayed on the display 12 through the output control unit 23 such that the normal region Tn and the ischemic region Ti are displayed in a superimposed manner on the medical image 40 in different manners. The display 12 shows an image display region 45 for displaying the medical image 40, an image information display field 46 for displaying image information of the medical image 40, a save button 47 for accepting saving of the medical image 40, and a delete button 48 for deleting the medical image 40 without saving the medical image 40. The image information to be displayed in the image information display field 46 includes, for example, a patient ID, an imaging date and time, the type of the organ portion T, the type of the medical imaging device 14, and so on. The save button 47 and the delete button 48 are operated by the user through the user interface 13.

As illustrated in FIG. 12, the medical image 40 to be displayed on the display 12 may be displayed such that only either the normal region In or the ischemic region Ti is displayed in a superimposed manner on the medical image 40. When the normal region In or the ischemic region Ti is divided into two separate regions in the organ portion T, the two regions are displayed in the same display manner. A medical image in which the organ portion T has either blood flow attribute has a low priority of being used for intestinal resection. Thus, when a plurality of medical images 40 are displayed sequentially, such a medical image may be displayed later than the medical image 40 having the normal region Tn and the ischemic region Ti (see FIG. 11).

In the display of the medical image 40, the normal region Tn and the ischemic region Ti may be visualized and displayed in the form of a schematic diagram, as well as in the form in which the normal region Tn and the ischemic region Ti are filled with colors. For example, the medical image editing unit 35 extracts the contour of an organ portion identified in the medical image 40 and the contour of a region recognized as the normal region In or the ischemic region Ti by the blood flow attribute recognition unit 34 to create a schematic diagram, and shows the schematic diagram in the image display region 45. The created schematic diagram and the medical image 40 may be displayed side by side.

When a plurality of search regions 42 are set in one medical image 40, it is preferable to infer, for each region, whether the blood flow attribute is different from or common to that of the reference region 41 to recognize a common attribute region. The respective evaluation results of the search regions 42 may be uniformly edited in the same way for each blood flow attribute and visualized, or the brightness or saturation may differ even for the same blood flow attribute. For example, a medical image 40 in which two normal regions In and two ischemic regions Ti are identified may be edited such that the normal regions Tn are filled with red color and orange color and the ischemic regions Ti are filled with blue color and light blue color.

The medical image 40 displayed on the display 12 may be stored in the main storage area 24 automatically rather than through the user's operation. The medical image 40 to be stored in the main storage area 24 also includes content visualized for the normal region In or the ischemic region Ti and the evaluation results, which are edited by the medical image editing unit 35. The medical image 40 to be stored automatically may be a result of determination of whether the medical image 40 is useful for intestinal resection. For example, the medical image 40 in which the organ portion T includes only the ischemic region Ti is not stored because of low importance for intestinal resection, and only a medical image having the normal region Tn and the ischemic region Ti is stored. Alternatively, the selection may be performed using the area ratio of the normal region In and the ischemic region Ti such that a medical image 40 in which the ischemic region Ti occupies a certain proportion or more is not stored.

The training of the classifier 33 will be described. An average pixel value in the normal region In or the ischemic region Ti of a learning image that is captured as a color image in advance and for which the blood flow attribute of the organ portion Tis acquired is calculated and input to the CNN 33a. The CNN 33a is trained to perform two-class classification to determine whether two regions in the medical image 40 during inference have the same blood flow attribute. The CNN 33a optimized by training outputs a probability that the reference region 41 and the search region 42 in the input medical image 40 have the same blood flow attribute. The classifier 33 having the optimized CNN 33a, that is, the trained classifier 33, performs two-class classification based on the output probability.

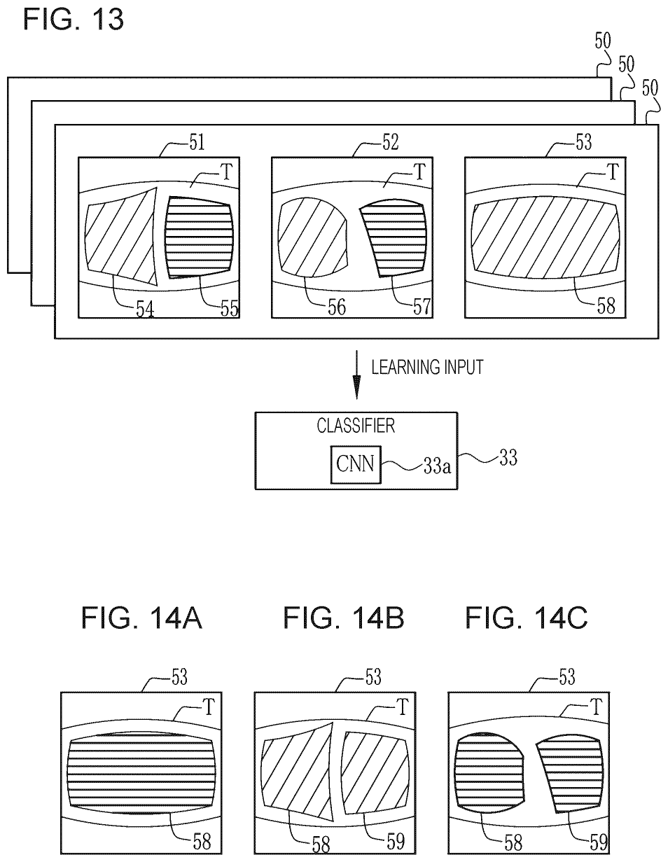

As illustrated in FIG. 13, when a triplet loss is used as a learning method, the CNN 33a is optimized by repeatedly inputting, for training, an input image set 50 that is a set of three learning images. The learning images of the input image set are each associated with at least one of the normal region Tn or the ischemic region Ti in advance. In the triplet loss, the input image set 50 constituted by three types of learning images, namely, an anchor image 51, a positive image 52, and a negative image 53, is input to the classifier 33 having the CNN 33a. The anchor image 51 is a learning image for reference input. The positive image 52 is a positive sample and is a learning image having a feature that, when used for inference, yields the same result as that of the anchor image 51. The negative image 53 is a negative sample and is a learning image having a feature that, when used for inference, yields a different classification result from those of the anchor image 51 and the positive image 52.

The blood flow attribute of the normal region In or the ischemic region Ti in a learning image is associated with an image region for each blood flow attribute by, for example, visual annotation or superimposition of a blood flow image after injection of a fluorescent agent using ICG fluorescence on the original image before injection.

During inference, the classifier 33 classifies a pattern in which the organ portion T in the medical image 40 has the normal region Tn and the ischemic region Ti and a pattern in which the organ portion T in the medical image 40 has only either the normal region Tn or the ischemic region Ti by using, for example, an image in which the organ portion T in the anchor image 51 has an anchor normal region 54 and an anchor ischemic region 55, the organ portion T in the positive image 52 has a positive normal region 56 and a positive ischemic region 57, and the organ portion T in the negative image 53 has a negative region 58 that is the normal region Tn or the ischemic region Ti.

As illustrated in FIGS. 14A to 14C, the negative images 53 in the input image sets 50 used for learning input include, in addition to the negative image 53 described with reference to FIG. 13 in which the negative region 58 is the normal region Tn, a negative image 53 (FIG. 14A) in which the negative region 58 is the ischemic region Ti, a negative image 53 (FIG. 14B) having a negative region 59 different from the negative region 58, each of the negative regions 58 and 59 being the normal region Tn, and a negative image 53 (FIG. 14C) having a negative region 59 different from the negative region 58, each of the negative regions 58 and 59 being the ischemic region Ti. The negative image 53 is not completely different from the anchor image 51 and the positive image 52, and is an image of a similar imaging target, such as an image of the same organ portion T of a different person or an image of a different organ portion T of the same person.

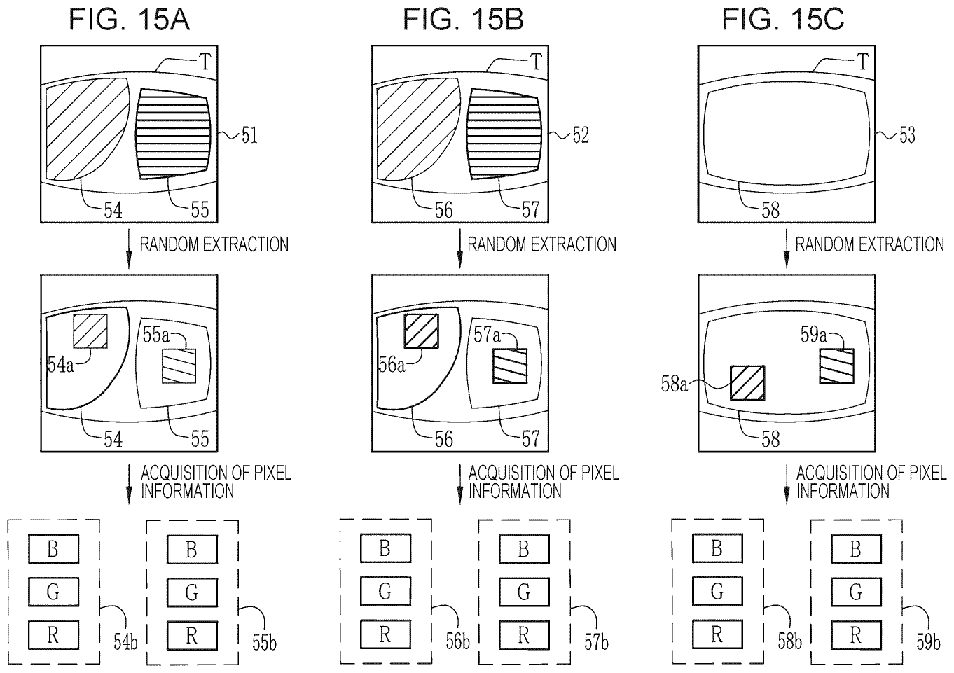

As illustrated in FIGS. 15A to 15C, the classifier 33 acquires pixel values of small regions randomly extracted from the normal region Tn and the ischemic region Ti in the organ portion T of each of the learning images constituting the input image set 50. The acquired pixel values are used to train and optimize the CNN 33a. The classifier 33 having the optimized CNN 33a is used for classification. From the anchor image 51, pixel information 54b in a small region 54a randomly extracted from the anchor normal region 54 and pixel information 55b in a small region 55a randomly extracted from the anchor ischemic region 55 are acquired (FIG. 15A). From the positive image 52, pixel information 56b in a small region 56a randomly extracted from the positive normal region 56 and pixel information 57b in a small region 57a randomly extracted from the positive ischemic region 57 are acquired (FIG. 15B). From the negative image 53, pixel information 58b in a small region 58a and pixel information 59b in a small region 59a, which are randomly extracted from the negative region 58, are acquired (FIG. 15C). The pieces of pixel information 54b to 59b each include pixel value distributions and average pixel values.

In the random extraction of the negative image 53 in FIG. 15C, when the organ portion T has only the negative region 58, the small region 58a and the small region 59a are extracted from the negative region 58, and when the organ portion T has the negative region 58 and the negative region 59 (FIGS. 14B and 14C), the small region 58a is extracted from the negative region 58, and the small region 59a is extracted from the negative region 59.

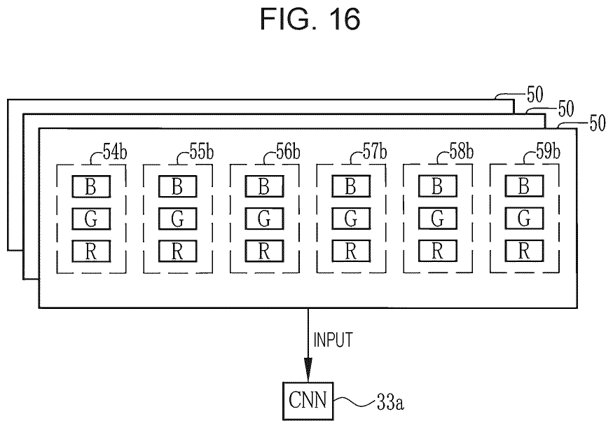

As illustrated in FIG. 16, in the classifier 33, the CNN 33a is trained using the pieces of pixel information 54b to 59b for the respective small regions extracted from the input image set 50. In training with the triplet loss, feature value conversion for converting pixel values into feature values is performed, and a loss calculated to ensure that the relationship between the feature values of two regions obtained by the conversion has a predetermined value is minimized. Through the training, the CNN 33a is optimized to perform conversion such that feature value distributions between regions having the same blood flow attribute are located at close positions in the feature value space and conversion such that feature value distributions between regions having different blood flow attributes are located at distant positions in the feature value space.

During inference in the CNN 33a, it is preferable that whether the blood flow attributes of the reference region 41 and the search region 42 in an input medical image 40 are the same or different be represented by a probability expressed as a numerical value greater than or equal to 0 and less than or equal to 1. For this reason, the CNN 33a is trained to optimize feature value conversion such that, in the medical image 40, feature value distributions overlap as much as possible in regions having the same blood flow attribute and feature value distributions do not overlap in regions having different blood flow attributes.

One method of the two-class classification processing is to extract color information such as pixel values of the reference region 41 and the search region 42 in the color space, convert the color information into feature values, calculate a probability by using a distance between two feature value distributions obtained by the conversion, and perform threshold value processing on the calculated probability. The distance between the representative values is subjected to threshold value processing to output an inference result.

The inference result is polarized into two patterns, namely, a pattern in which the blood flow attributes are common and a pattern in which the blood flow attributes are different, based on pixel values extracted from two regions in the medical image 40. For this reason, the feature value conversion converts feature value distributions into distributions biased more than pixel value distributions. The CNN 33a can be trained using a distance between regions using representative values such as average feature values. The representative values may be median values or the like instead of the average values of the feature values. Instead of a probability being calculated, the rate of match between the feature value distributions may be calculated and output as an inference result.

Specifically, in the anchor image 51, a distance D1 between an average feature value obtained by converting the pixel information 54b in the anchor normal region 54 and an average feature value obtained by converting the pixel information 55b in the anchor ischemic region 55 is calculated. Likewise, a distance D2 between the average feature value of the positive normal region 56 and the average feature value of the positive ischemic region 57 in the positive image 52 and a distance D3 between the average feature value of the negative region 58 and the average feature value of the negative region 59 in the negative image 53 are calculated. In this case, when the difference between the distance D1 and the distance D2 is represented by a distance Dp and the difference between the distance D1 and the distance D3 is represented by a distance Dn, the CNN 33a is optimized such that the distance Dp is as close to 0 as possible and the distance Dn has a large value.

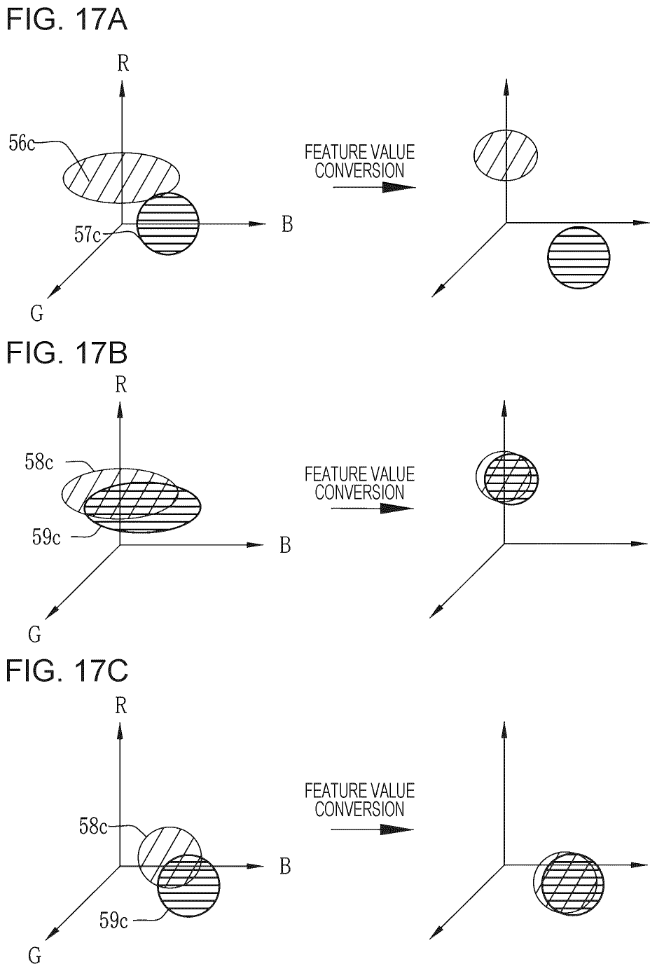

As illustrated in FIGS. 17A to 17C, the optimized CNN 33a performs feature value conversion on pixel distributions included in each piece of pixel information. The CNN 33a performs feature value conversion on a pixel distribution 56c in the normal region Tn and a pixel distribution 57c in the ischemic region Ti of the positive image 52 such that the feature value distributions are away in the feature value space (FIG. 17A), and performs feature value conversion on a pixel distribution 58c and a pixel distribution 59c in the negative image 53 such that the feature value distributions are located at close positions in the feature value space when the pixel distributions 58c and 59c are in the normal regions In (FIG. 17B) and the feature value distributions are also located at close positions in the feature value space when the pixel distributions 58c and 59c are in the ischemic regions Ti (FIG. 17C). The CNN 33a is repeatedly trained to perform such feature value conversion.

During inference, the pixel values (the B pixel value, the G pixel value, and the R pixel value) of each of the reference region 41 and the search region 42 in the medical image 40 are input to the CNN 33a, and a probability that the blood flow attributes (normal or ischemic) are equal is output. For example, an inference result having a value greater than or equal to 0 and less than or equal to 1 is output, and the output value is recognized to classify the relationship between the blood flow attributes. The threshold value for classification is preferably set to 0.5. When the output value is greater than or equal to 0.5, the attributes of the two regions are classified as being the same, and when the output value is less than 0.5, the attributes of the two regions are classified as being different. In learning, the CNN 33a is trained so that the probability to be output as an inference result is biased to around 0 or 1 (e.g., so that only either a value greater than or equal to 0 and less than or equal to 0.1 or a value greater than or equal to 0.9 and less than or equal to 1 is output).

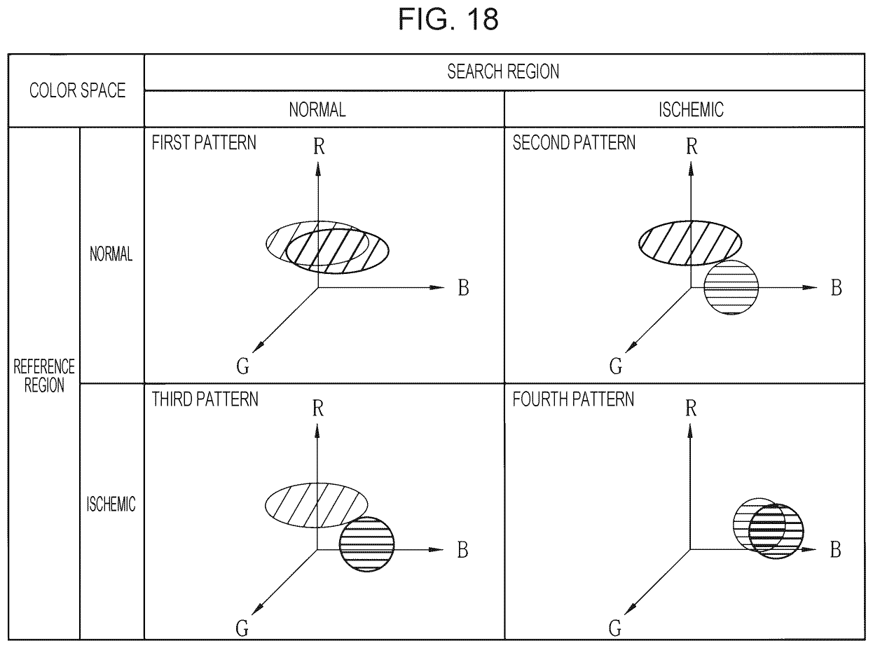

As illustrated in FIG. 18, medical images 40 in which the reference region 41 and the search region 42 are set in the organ portion T are represented as the following four patterns in a color space on the basis of the relationship of color information. A medical image 40 in any one of a first pattern in which the reference region 41 and the search region 42 are normal, a second pattern in which the reference region 41 is normal and the search region 42 is ischemic, a third pattern in which the reference region 41 is ischemic and the search region 42 is normal, and a fourth pattern in which the reference region 41 and the search region 42 are ischemic is input to the classifier 33.

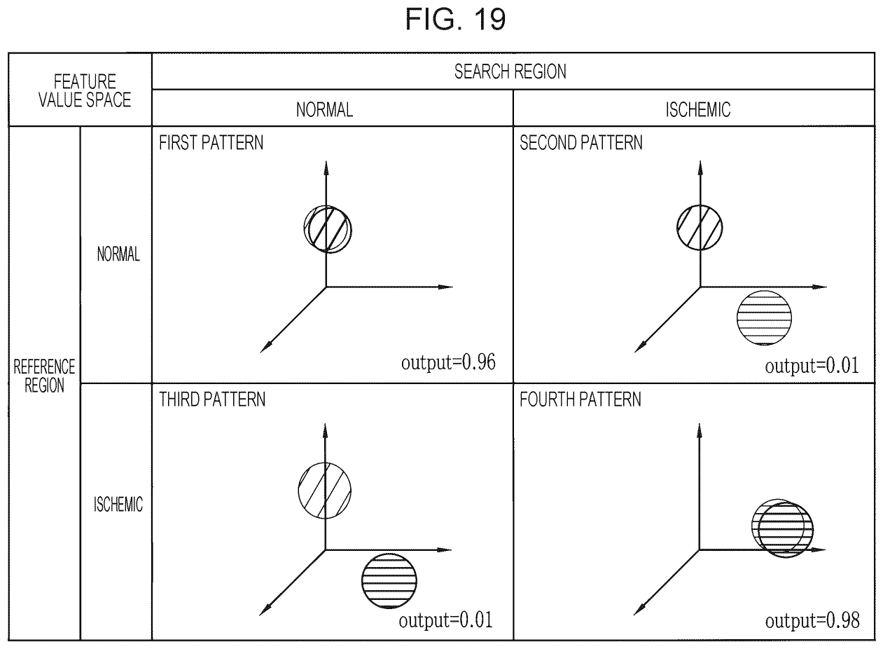

As illustrated in FIG. 19, the classifier 33 performs two-class classification for determining whether the reference region 41 and the search region 42 in the medical image 40 have the same blood flow attribute, by using the CNN 33a, through inference based on a relationship obtained through conversion into feature value distributions. As a result of the inference, for example, as the first to fourth patterns described above, a probability that the reference region 41 and the search region 42 have the same blood flow attribute is output from the CNN 33a in the following way. The classifier 33 applies a threshold value to the probability that the positional relationship between the two output feature value distributions is the same to classify the relationship between the blood flow attributes. A value of 0.96 is output for a medical image 40 in the first pattern, and the medical image 40 is classified as having a common blood flow attribute. A value of 0.01 is output for a medical image 40 in the second pattern, and the medical image 40 is classified as having different blood flow attributes. A value of 0.01 is output for a medical image 40 in the third pattern, and the medical image 40 is classified as having different blood flow attributes. A value of 0.98 is output for a medical image 40 in the fourth pattern, and the medical image 40 is classified as having a common blood flow attribute. That is, on the basis of the relationship of color information, the two regions in the first pattern and the fourth pattern are classified as having the same attribute, and the two regions in the second pattern and the third pattern are classified as having different attributes.

Since the classifier 33 performs only classification of the relationship between the blood flow attributes of the reference region 41 and the search region 42, as described above, after the classification, the classification result is associated with the medical image 40, and the medical image 40 is transmitted to the blood flow attribute recognition unit 34. The blood flow attribute recognition unit 34 identifies the blood flow attributes of the reference region 41 and the search region 42.

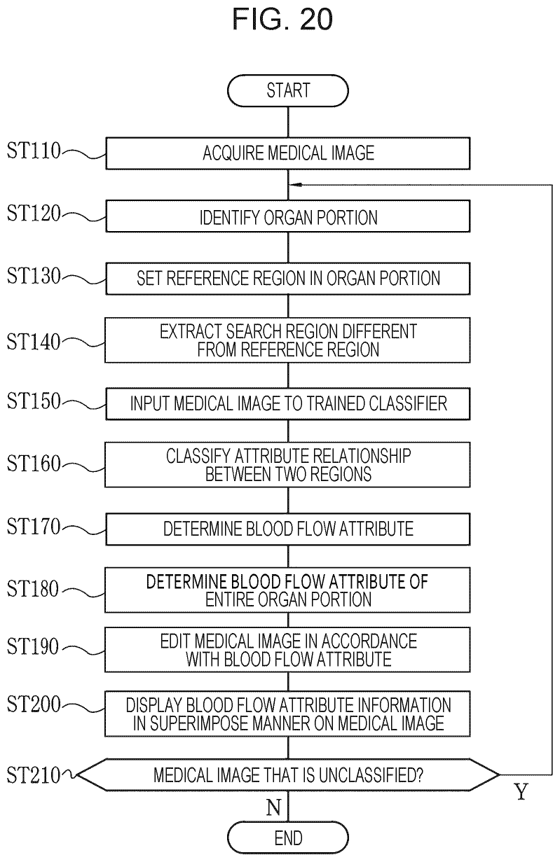

The flow of a series of operations for controlling classification of a medical image 40 according to a blood flow attribute in the medical image recognition system 10 according to the present embodiment will be described with reference to a flowchart illustrated in FIG. 20. The medical image recognition apparatus 11 acquires a medical image 40 obtained by imaging the organ portion T in the living body with the medical imaging device 14 (step ST110). In the medical image 40 input to the blood flow information recognition unit 30, the organ portion T is identified by the organ portion identification unit 31 (step ST120). A reference region in the organ portion T is set (step ST130). A search region different from the reference region is comprehensively extracted (step ST140). The medical image 40 in which the reference region 41 and the search region 42 are set is input to the trained classifier 33 (step ST150).

The classifier 33 performs two-class classification for classifying whether the blood flow attributes of the reference region 41 and the search region 42 are common or different by using the CNN 33a (step ST160). In the two-class classification, the blood flow attribute of either the reference region 41 or the search region 42 is determined, and the blood flow attribute of the other region is identified (step ST170). In the organ portion T, a region having a blood flow attribute equal to the determined blood flow attribute is recognized (step ST180). The medical image editing unit 35 performs editing to visualize the blood flow attribute determined in the organ portion T of the medical image 40 (step ST190). The display 12 displays the medical image 40 with superimposed blood flow information (step ST200). If there is a medical image 40 that is unclassified for the blood flow attribute (Y in step ST210), the medical image 40 is input to the blood flow information recognition unit 30 to perform blood flow information classification processing on the organ portion T (step ST120). If there is no medical image 40 that is unclassified (N in step ST210), the medical image 40 displayed with the superimposed blood flow information is stored, and the series of operations ends.

When the classifier 33 classifies the relationship between the blood flow attributes of the reference region 41 and the search region 42 in medical images 40, only a medical image 40 in which the blood flow attributes are different may be transmitted to the blood flow attribute recognition unit 34. That is, only a medical image 40 in which a demarcation line can be set is selected. This can reduce the load or processing time on the blood flow information recognition unit 30.

In the embodiment described above, the hardware structures of processing units that perform various processes, such as the central control unit, the image acquisition unit 21, the input receiving unit 22, and the output control unit 23 implemented in the medical image recognition apparatus 11, and the organ portion identification unit 31, the region setting unit 32, the classifier 33, the blood flow attribute recognition unit 34, and the medical image editing unit 35 included in the blood flow information recognition unit 30, are various processors as follows. The various processors include a central processing unit (CPU), which is a general-purpose processor executing software (program) to function as various processing units, a programmable logic device (PLD) such as a field programmable gate array (FPGA), which is a processor whose circuit configuration is changeable after manufacturing, a dedicated electric circuit that is a processor having a circuit configuration specifically designed to execute various types of processing, and so on.

A single processing unit may be configured as one of the various processors or as a combination of two or more processors of the same type or different types (e.g., a plurality of FPGAs or a combination of a CPU and an FPGA). Alternatively, a plurality of processing units may be configured as a single processor. Examples of configuring a plurality of processing units as a single processor include, first, a form in which, as typified by a computer such as a client or a server, the single processor is configured as a combination of one or more CPUs and software and the processor functions as the plurality of processing units. The examples include, second, a form in which, as typified by a system on chip (SoC) or the like, a processor is used in which the functions of the entire system including the plurality of processing units are implemented as one integrated circuit (IC) chip. As described above, the various processing units are configured by using one or more of the various processors described above as a hardware structure.

More specifically, the hardware structure of these various processors is an electric circuit (circuitry) in which circuit elements such as semiconductor elements are combined. The hardware structure of a storage unit is a storage device such as a hard disc drive (HDD) or a solid state drive (SSD).

From the foregoing description, a medical image recognition apparatus, an endoscope system, or a method for operating the medical image recognition apparatus according to Appendices 1 to 15 below can be understood.

### APPENDIX 1

A medical image recognition apparatus including a processor configured to:
acquire a medical image;
set a reference region and an evaluation region different from the reference region in an organ portion of the medical image;
input color information of the reference region and color information of the evaluation region to a trained classifier;
output an evaluation result of a blood flow attribute of the evaluation region with respect to a blood flow attribute of the reference region; and
perform control to display an evaluation result of the blood flow attribute of the evaluation region.

### APPENDIX 2

The medical image recognition apparatus according to Appendix 1, wherein the processor is configured to set the reference region by using at least one of manual setting by a user or automatic recognition.

### APPENDIX 3

The medical image recognition apparatus according to Appendix 1 or 2, wherein the processor is configured to:
recognize that in the medical image, a region for which a difference from the color information of the reference region is within a certain range has a blood flow attribute equal to the blood flow attribute of the reference region; and
recognize that in the medical image, a region for which a difference from the color information of the evaluation region is within a certain range has a blood flow attribute equal to the blood flow attribute of the evaluation region.

### APPENDIX 4

The medical image recognition apparatus according to any one of Appendices 1 to 3, wherein the processor is configured to:
calculate, for the medical image input to the trained classifier, a probability that the blood flow attribute of the reference region and the blood flow attribute of the evaluation region are equal; and
output that the blood flow attribute of the reference region and the blood flow attribute of the evaluation region are equal in a case where the probability is greater than or equal to a preset threshold value.

### APPENDIX 5

The medical image recognition apparatus according to any one of Appendices 1 to 4, wherein the processor is configured to:
extract pixel values of the reference region and the evaluation region as the color information; and
output an evaluation result acquired by converting the pixel values into feature values.

### APPENDIX 6

The medical image recognition apparatus according to any one of Appendices 1 to 5, wherein the processor is configured to:
apply a grid to the medical image; and
set the evaluation region from regions segmented by the grid.

### APPENDIX 7

The medical image recognition apparatus according to any one of Appendices 1 to 6, wherein the processor is configured to visualize and display the blood flow attribute of the reference region together with the evaluation result of the blood flow attribute of the evaluation region.

### APPENDIX 8

The medical image recognition apparatus according to any one of Appendices 1 to 7, wherein the processor is configured to set the reference region and the evaluation region in one medical image.

### APPENDIX 9

The medical image recognition apparatus according to any one of Appendices 1 to 8, wherein the processor is configured to:
set a plurality of the evaluation regions from one medical image; and
display evaluation results of blood flow attributes of the plurality of the evaluation regions.

### APPENDIX 10

The medical image recognition apparatus according to any one of Appendices 1 to 9, wherein the processor is configured to determine whether the evaluation region is a normal region in which blood flow is normal or an ischemic region in which blood flow is ischemic, by using the blood flow attribute of the reference region as a reference.

### APPENDIX 11

The medical image recognition apparatus according to Appendix 10, wherein the processor is configured to use the trained classifier trained using learning images associated with an image region for each blood flow attribute.

### APPENDIX 12

The medical image recognition apparatus according to Appendix 11, wherein the processor is configured to use the trained classifier trained using average pixel values of small regions randomly extracted from the normal region and the ischemic region in the learning image.

### APPENDIX 13

The medical image recognition apparatus according to any one of Appendices 10 to 12, wherein the processor is configured to display the medical image having the normal region and the ischemic region in preference to the medical image having either the normal region or the ischemic region.

### APPENDIX 14

An endoscope system including:
the medical image recognition apparatus according to Appendix 1;
an endoscope that acquires the medical image;
a display that displays the medical image and the evaluation result of the blood flow attribute of the evaluation region; and
user input means for accepting manual setting by a user.

### APPENDIX 15

A method for operating a medical image recognition apparatus, including:
a step of acquiring a medical image;
a step of setting a reference region and an evaluation region different from the reference region in an organ portion of the medical image;
a step of inputting color information of the reference region and color information of the evaluation region to a trained classifier;
a step of outputting an evaluation result of a blood flow attribute of the evaluation region with respect to a blood flow attribute of the reference region; and
a step of performing control to display an evaluation result of the blood flow attribute of the evaluation region.

### REFERENCE SIGNS LIST

10 medical image recognition system
11 medical image recognition apparatus
12 display
13 user interface
14 medical imaging device
21 image acquisition unit
22 input receiving unit
23 output control unit
24 main storage area
30 blood flow information recognition unit
31 organ portion identification unit
32 region setting unit
33 classifier
33a CNN
34 blood flow attribute recognition unit
35 medical image editing unit
40 medical image
41 reference region
42 search region
45 image display region
46 image information display field
47 save button
48 delete button
50 input image set
51 anchor image
52 positive image
53 negative image
54 anchor normal region
55 anchor ischemic region
56 positive normal region
57 positive ischemic region
58 negative region
59 negative region
54A to 59A small region
54B to 59B pixel information
56C to 59C pixel distribution
Gr grid
H photographic subject
ST step
T organ portion
Ti ischemic region
Tn normal region

What is claimed is:

1. A medical image recognition apparatus comprising a processor configured to:
acquire a medical image;
set a reference region and an evaluation region different from the reference region in an organ portion of the medical image;
input color information of the reference region and color information of the evaluation region to a trained classifier;
output an evaluation result of a blood flow attribute of the evaluation region with respect to a blood flow attribute of the reference region; and
perform control to display an evaluation result of the blood flow attribute of the evaluation region.

2. The medical image recognition apparatus according to claim 1, wherein the processor is configured to set the reference region by using at least one of manual setting by a user or automatic recognition.

3. The medical image recognition apparatus according to claim 1, wherein the processor is configured to:
recognize that in the medical image, a region for which a difference from the color information of the reference region is within a certain range has a blood flow attribute equal to the blood flow attribute of the reference region; and
recognize that in the medical image, a region for which a difference from the color information of the evaluation region is within a certain range has a blood flow attribute equal to the blood flow attribute of the evaluation region.

4. The medical image recognition apparatus according to claim 3, wherein the processor is configured to:
calculate, for the medical image input to the trained classifier, a probability that the blood flow attribute of the reference region and the blood flow attribute of the evaluation region are equal; and
output that the blood flow attribute of the reference region and the blood flow attribute of the evaluation region are equal in a case where the probability is greater than or equal to a preset threshold value.

5. The medical image recognition apparatus according to claim 4, wherein the processor is configured to:
extract pixel values of the reference region and the evaluation region as the color information; and
output an evaluation result acquired by converting the pixel values into feature values.

6. The medical image recognition apparatus according to claim 1, wherein the processor is configured to:
apply a grid to the medical image; and
set the evaluation region from regions segmented by the grid.

7. The medical image recognition apparatus according to claim 1, wherein the processor is configured to visualize and display the blood flow attribute of the reference region together with the evaluation result of the blood flow attribute of the evaluation region.

8. The medical image recognition apparatus according to claim 7, wherein the processor is configured to set the reference region and the evaluation region in one medical image.

9. The medical image recognition apparatus according to claim 1, wherein the processor is configured to:
set a plurality of the evaluation regions from one medical image; and
display evaluation results of blood flow attributes of the plurality of the evaluation regions.

10. The medical image recognition apparatus according to claim 1, wherein the processor is configured to determine whether the evaluation region is a normal region in which blood flow is normal or an ischemic region in which blood flow is in an ischemic state, by using the blood flow attribute of the reference region as a reference.

11. The medical image recognition apparatus according to claim 10, wherein the processor is configured to use the trained classifier trained using learning images associated with an image region for each blood flow attribute.

12. The medical image recognition apparatus according to claim 11, wherein the processor is configured to use the trained classifier trained using average pixel values of small regions randomly extracted from the normal region and the ischemic region in the learning image.

13. The medical image recognition apparatus according to claim 12, wherein the processor is configured to display the medical image having the normal region and the ischemic region in preference to the medical image having either the normal region or the ischemic region.

14. An endoscope system comprising:
the medical image recognition apparatus according to claim 1;
an endoscope that acquires the medical image;
a display that displays the medical image and the evaluation result of the blood flow attribute of the evaluation region; and
user input means for accepting manual setting by a user.

15. A method for operating a medical image recognition apparatus, comprising:
a step of acquiring a medical image;
a step of setting a reference region and an evaluation region different from the reference region in an organ portion of the medical image;
a step of inputting color information of the reference region and color information of the evaluation region to a trained classifier;
a step of outputting an evaluation result of a blood flow attribute of the evaluation region with respect to a blood flow attribute of the reference region; and
a step of performing control to display an evaluation result of the blood flow attribute of the evaluation region.
